(19) United States
(12) Patent Application Publication
(10) Pub. No.: US 2025/0049811 A1
(43) Pub. Date: Feb. 13, 2025

(54) METHODS OF TREATING PANCREATIC CANCER BY USING BET INHIBITOR AND PARP INHIBITOR

(71) Applicant: NATIONAL CHENG KUNG UNIVERSITY, Tainan City (TW)

(72) Inventors: Yan-Shen SHAN, Tainan City (TW); Wen-Pin SU, Tainan City (TW); Ya-Chin HOU, Tainan City (TW)

(21) Appl. No.: 18/523,965

(22) Filed: Nov. 30, 2023

(30) Foreign Application Priority Data
Aug. 10, 2023 (TW) 112130137

(51) Int. Cl. A61K 31/55; A61K 31/437; A61P 35/00

(52) U.S. Cl. CPC A61K 31/55; A61K 31/437; A61P 35/00

(57) ABSTRACT

Disclosed herein is a method of treating a pancreatic cancer in a subject who is responsive to a platinum-based therapy. The method comprises administering to the subject an effective amount of a BET inhibitor and an effective amount of a PARP inhibitor. According to some embodiments of the present disclosure, the BET inhibitor is mivebresib, JQ1, or AZD5153, and the PARP inhibitor is rucaparib, veliparib, or olaparib.

### CROSS-REFERENCE TO RELATED APPLICATIONS

This application relates to and claims the benefit of TW Application No. 112130137, filed Aug. 10, 2023; the content of the application is incorporated herein by reference in its entirety.

### BACKGROUND OF THE INVENTION

### 1. Field of the Invention

The present disclosure in general relates to the field of cancer treatment. More particularly, the present disclosure relates to methods of treating pancreatic cancers by using an inhibitor of bromodomain and extra-terminal protein (BET) and an inhibitor of poly ADP-ribose polymerase (PARP).

### 2. Description of Related Art

Pancreatic cancer is the fourth leading cause of cancer death worldwide. Most patients are diagnosed at late stage, due to the fact that pancreatic cancer usually shows little or no symptoms until it has advanced or spread. Further, over 80% of patients develop disease recurrence after resection of pancreatic cancer. Accordingly, pancreatic cancer has the lowest survival of all common cancers, with an average 5-year survival rate of about 10%; and the treatment of pancreatic cancer remains a formidable challenge. Pancreatic ductal adenocarcinoma (PDAC) is the most prevalent malignancy of the pancreas that accounts for more than 85-90% of pancreatic cancer cases, with an average 5-year survival rate of less than 10%. In the United States, the incidence of pancreatic cancer is rising at a rate of 0.5% to 1% per year. The risk factors associated with pancreatic cancer include age, smoking, obesity, diabetes, and chronic pancreatitis. It is reported that the incidence and mortality of pancreatic cancer in developed countries are higher than that of developing countries, and there is a positive correlation between pancreatic mortality and human development index (HDI).

Surgery is typically the main treatment for pancreatic cancer. However, about 80% of patients who underwent surgery would relapse and require the further treatment of chemotherapy (also known as “adjuvant chemotherapy”). Gemcitabine and capecitabine are commonly used as adjuvant chemotherapy. FOLFIRINOX regimen (the combined treatment of fluorouracil (5-FU), leucovorin, irinotecan, and oxaliplatin) provides an alternative means to improve the survival outcomes for patients with pancreatic cancer. Unfortunately, these treatments usually lead to serious side effects, and are only suitable for some patients. Further, the development of drug resistance continues to be a major problem in the effective treatment of pancreatic cancer.

In view of the foregoing, there is a continuing interest in developing a novel agent and/or method for treating pancreatic cancer.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the present disclosure is directed to a method of treating a pancreatic cancer in a subject. The method comprises administering to the subject an effective amount of a BET inhibitor and an effective amount of a PARP inhibitor, thereby alleviating or ameliorating the symptoms associated with the pancreatic cancer. According to certain embodiments of the present disclosure, the subject is response to a platinum-based therapy.

According to some embodiments of the present disclosure, the BET inhibitor is mivebresib, JQ1, or AZD5153; and the PARP inhibitor is rucaparib, veliparib, or olaparib. In certain exemplary embodiments, the BET inhibitor is mivebresib, and the PARP inhibitor is rucaparib. Preferably, the BET inhibitor (e.g., mivebresib) and the PARP inhibitor (e.g., rucaparib) are administered to the subject at a weight ratio of 1:10.

According to some embodiments, the platinum-based therapy comprises the treatment of cisplatin, carboplatin, oxaliplatin, or nedaplatin. In certain exemplary embodiments, the platinum-based therapy comprises the treatment of cisplatin.

The second aspect of the present disclosure pertains to a pharmaceutical composition or kit for treating a pancreatic cancer in a subject who is response to a platinum-based therapy.

According to some embodiments, the pharmaceutical composition or kit comprises an effective amount of a BET inhibitor and an effective amount of a PARP inhibitor.

Preferably, the BET inhibitor and PARP inhibitor are present in the pharmaceutical composition at a weight ratio of 1:10.

The subject is a mammal; preferably, a human.

Many of the attendant features and advantages of the present disclosure will becomes better understood with reference to the following drawings and detailed description.

### BRIEF DESCRIPTION OF THE DRAWINGS

The present description will be better understood from the following detailed description read in light of the accompanying drawings briefly discussed below.

FIGS. 1A and 1B are histograms respectively depicting the inhibitory effect of mivebresib and/or rucaparib on pancreatic cell lines MIA-PaCa2 (FIG. 1A) and Panc-1 (FIG. 1B) according to Example 1 of the present disclosure. **p≤0.01, ***p≤0.001. Combine: combined treatment of mivebresib and rucaparib.

FIG. 2 is a line chart depicting the anti-tumor effect of mivebresib and/or rucaparib on subcutaneous tumor model according to Example 2 of the present disclosure. **p≤0.01 ***p≤0.001. Combine: combined treatment of mivebresib and rucaparib.

FIG. 3 is a histogram depicting the anti-tumor effect of mivebresib and/or rucaparib on orthotropic tumor model according to Example 2 of the present disclosure. *p≤0.05. Combine: combined treatment of mivebresib and rucaparib.

FIG. 4 is a histogram depicting the expression level of BIM in the patients responsive (responders) or unresponsive (non-responders) to the platinum-based therapy according to Example 4 of the present disclosure. **p≤0.01. FPKM: fragments per kilobase per million.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “treat,” “treating” and “treatment” are interchangeable, and encompasses partially or completely preventing, ameliorating, mitigating and/or managing a symptom, a secondary disorder or a condition associated with pancreatic cancer. The term “treating” as used herein refers to application or administration of the BET inhibitor and PARP inhibitor of the present disclosure to a subject, who has a symptom, a secondary disorder or a condition associated with pancreatic cancer, with the purpose to partially or completely alleviate, ameliorate, relieve, delay onset of, inhibit progression of, reduce severity of, and/or reduce incidence of one or more symptoms, secondary disorders or features associated with pancreatic cancer. Symptoms, secondary disorders, and/or conditions associated with pancreatic cancer include, but are not limited to, jaundice, blood clots, belly or back pain, weight loss, poor appetite, diarrhea, nausea, vomiting, fatigue, bloating, diabetes, or a combination thereof. Treatment may be administered to a subject who exhibits only early signs of such symptoms, disorder, and/or condition for the purpose of decreasing the risk of developing the symptoms, secondary disorders, and/or conditions associated with pancreatic cancer. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Persons having ordinary skills could calculate the human equivalent dose (HED) for the medicament (such as the BET inhibitor and PARP inhibitor of the present disclosure) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “administered”, “administering” or “administration” are used interchangeably herein to refer a mode of delivery, including, without limitation, orally, subcutaneously, intratumorally, intravenously, intraperitoneally, or intraarterially administering an agent (for example, the present BET inhibitor and PARP inhibitor). In certain embodiments of the present disclosure, the BET inhibitor and PARP inhibitor are orally delivered to the subject so as to produce a therapeutic effect in the subject.

As used herein, a subject being responsive to an agent (e.g., a platinum-based therapy) indicates that the agent, after being administered to the subject, is capable of inhibiting or alleviating the progress of a disease (for example, tumor growth), thereby ameliorating the symptom, secondary disorder or condition associated with the disease in the subject.

The phrase “pharmaceutically acceptable” refers to molecular entities and compositions that are “generally regarded as safe,” e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “subject” refers to a mammal including the human species that is treatable with the BET inhibitor and PARP inhibitor, pharmaceutical composition, kit, and/or method of the present disclosure. The term “subject” is intended to refer to both the male and female gender unless one gender is specifically indicated.

### II. Description of the Invention

The present disclosure is based, at least in part, on the discovery that the BET inhibitor and PARP inhibitor exhibit a synergistic effect on inhibiting the growth of pancreatic cancer; thus, the combined therapy is useful in treating pancreatic cancer patients who develop resistance to a platinum-based therapy. Accordingly, the present disclosure provides a kit or pharmaceutical composition comprising a BET inhibitor and a PARP inhibitor, and the use of the kit or pharmaceutical composition in treating pancreatic cancer.

### (i) Kit or Pharmaceutical Composition

The first aspect of the present disclosure is directed to a kit or pharmaceutical composition comprising a BET inhibitor and a PARP inhibitor.

According to some embodiments of the present disclosure, the BET inhibitor is mivebresib, JQ1 (having the structure of

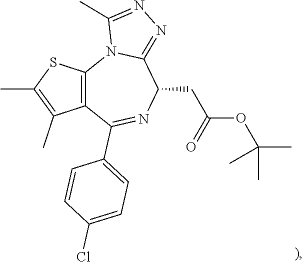 [SMILES: Cc1sc2c(c1C)C(c1ccc(Cl)cc1)=N[C@@H](CC(=O)OC(C)(C)C)c1nnc(C)n1-2]

or AZD5153 (having the structure of

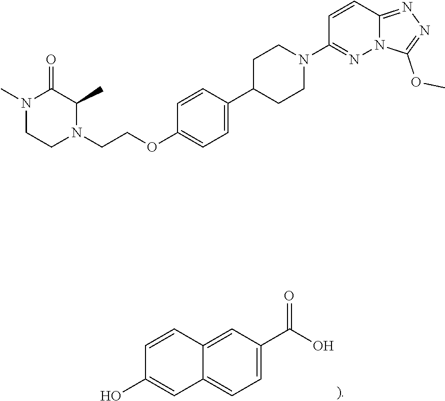

According to some embodiments of the present disclosure, the PARP inhibitor is rucaparib, veliparib, or olaparib. In certain preferred embodiments, the BET inhibitor is mivebresib, and the PARP inhibitor is rucaparib.

Depending on intended purpose, the BET inhibitor and PARP inhibitor may be formulated into a pharmaceutical composition. Alternatively, the BET inhibitor and PARP inhibitor may be formulated individually, and then presented as a pharmaceutical kit. Accordingly, in certain embodiments, in addition to the BET inhibitor and PARP inhibitor, the present pharmaceutical composition or kit further comprises a pharmaceutically acceptable carrier or excipient; in this case, the BET inhibitor and PARP inhibitor can be formulated, together with the pharmaceutically acceptable carrier or excipient, into solid, semi-solid, or liquid forms, such as powders, tablets, capsules, troches, pastes, granules, and ointments. As such, the administration of the present active components (i.e., the BET inhibitor and PARP inhibitor) can be achieved in various ways, including oral, buccal, topical, parenteral, and etc. administration. Exemplary formulations suitable for oral administration include, but are not limited to, powders, tablets, capsules (e.g., elastic gelatin capsules), troches, suspensions (e.g., aqueous or non-aqueous liquid suspensions, oil-in-water emulsions, or water-in-oil emulsions), dispersions, solutions, and elixirs. Exemplary formulations suitable for parenteral administration include, but are not limited to, isotonic suspensions, solutions, and emulsions in oily or aqueous vehicles.

Optionally, applicable solid carriers may include one or more substances that may also act as flavoring agents, lubricants, solubilizers, suspending agents, fillers, glidants, compression aids, binders or tablet-disintegrating agents, or encapsulating materials. In powders, the carrier is a finely divided solid that is in admixture with the finely divided active ingredient. In tablets, the active ingredient is mixed with an carrier having the necessary compression properties in suitable proportions and compacted in the shape and size desired. The powders and tablets preferably contain up to 99% of the active ingredient. Suitable solid carrier includes, for example, calcium phosphate, magnesium stearate, talc, sugars, lactose, dextrin, starch, gelatin, cellulose, methyl cellulose, sodium carboxymethyl cellulose, polyvinylpyrrolidine and the like.

When the present BET inhibitor and PARP inhibitor are formulated to be administered by intravenous, intraarterial, intratumoral, intraperitoneal, mucosal, or subcutaneous injection, the BET inhibitor and PARP inhibitor are in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, intraarterial, intratumoral, intraperitoneal, mucosal, or subcutaneous injection should contain, in addition to the present BET inhibitor and PARP inhibitor, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the invention may also contain stabilizers, preservatives, buffers, antioxidants, or other additives known to those of skill in the art.

The pharmaceutical composition or kit is prepared in accordance with acceptable pharmaceutical procedures, such as described in Remington's Pharmaceutical Sciences, 17^{th} edition, Alfonoso R. Gennaro, Mack Publishing Company, Easton, Pa (1985). Pharmaceutically acceptable carriers are those that are compatible with other ingredients in the formulation and biologically acceptable.

According to some embodiments, the BET inhibitor and PARP inhibitor are present in the present pharmaceutical composition at a weight ratio ranging from 1:0.1 to 1:100; for example, about 1:0.1, 1:0.2, 1:0.3, 1:0.4, 1:0.5, 1:0.6, 1:0.7, 1:0.8, 1:0.9, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100. In one preferred embodiments, the BET inhibitor and PARP inhibitor are present in the present pharmaceutical composition at a weight ratio of 1:10.

### (ii) Methods of Treating Pancreatic Cancer by Using BET Inhibitor and PARP Inhibitor

The second aspect of the present disclosure pertains to a method of treating a pancreatic cancer in a subject. The method comprises administered to the subject an effective amount of a BET inhibitor and an effective amount of a PARP inhibitor, or an effective amount of the pharmaceutical composition as described in section (i) of the present disclosure. According to some embodiments of the present disclosure, the subject is responsive to a platinum-based therapy, i.e., responsive to the treatment of cisplatin, carboplatin, oxaliplatin or nedaplatin. In one specific embodiment, the subject is responsive to the treatment of cisplatin.

As described above, the BET inhibitor may be mivebresib, JQ1, or AZD5153; and the PARP inhibitor may be rucaparib, veliparib, or olaparib. In certain preferred embodiments, the BET inhibitor is mivebresib, and the PARP inhibitor is rucaparib.

Depending on desired purpose, the BET inhibitor and PARP inhibitor are administered to the subject at a weight ratio ranging from 1:0.1 to 1:100. According to one preferred embodiment, the BET inhibitor and PARP inhibitor are administered to the subject at a weight ratio 1:10. In the embodiment, the BET inhibitor is mivebresib, and the PARP inhibitor is rucaparib.

According to some embodiments of the present disclosure, the subject is a mouse. In the embodiment, the effective amount of the BET inhibitor is about 0.01 to 100 mg/Kg body weight of the subject (such as, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/Kg body weight of the subject); and the effective amount of the PARP inhibitor is about 0.1 to 1,000 mg/Kg body weight of the subject (such as, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/Kg body weight of the subject). Preferably, the effective amount of the BET inhibitor is about 0.1 to 10 mg/Kg body weight of the subject, and the effective amount of the PARP inhibitor is about 1 to 100 mg/Kg body weight of the subject. In one exemplary embodiment, 1 mg/Kg of the BET inhibitor (e.g., mivebresib) and 10 mg/Kg of the PARP inhibitor (e.g., rucaparib) are sufficient to inhibit the tumor growth in the subject.

A skilled artisan may calculate the human equivalent dose (HED) for the medicament (i.e., the present BET inhibitor and PARP inhibitor) based on the doses determined from animal models. For example, the HED of the BET inhibitor is about 1 g/Kg body weight of the subject to about 10 mg/Kg body weight of the subject (such as, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, or 950 g/Kg body weight of the subject; or 1, 2, 3, 4, 5, 6, 7, 8, 9, or 10 mg/Kg body weight of the subject); and the HED of the PARP inhibitor is about 10 g/Kg body weight of the subject to about 100 mg/Kg body weight of the subject (such as, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, or 950 g/Kg body weight of the subject; or 1, 2, 3, 4, 5, 6, 7, 8, 9, 10, 15, 20, 25, 30, 35, 40, 45, 50, 55, 60, 65, 70, 75, 80, 85, 90, 95, or 100 mg/Kg body weight of the subject). The dose can be administered in a single aliquot, or alternatively in more than one aliquot. A skilled artisan or clinical practitioner may adjust the dosage or regime in accordance with the physical condition of the patient or the severity of the diseases.

As could be appreciated, the present BET inhibitor and PARP inhibitor may be administered to the subject in single one dosage or multiple dosages. For example, during the term of the present treatment, the active agent (i.e., the BET inhibitor and PARP inhibitor) may be administered to the subject once; alternatively, the active agent (i.e., the BET inhibitor and PARP inhibitor) may be administered to the subject several times, e.g., once every day, once every week, two times every week, three times every week, four times every week, five times every week, six times every week, once every two weeks, three times every two weeks, five times every two weeks, once every month, two times every month, three times every month, or longer, until sufficient therapeutic levels are achieved. The dosing regimen can be vary with factors such as the severity of the disease and the condition of the subject. A skilled artisan or clinical practitioner may determine the dosing regimen according to the clinical conditions.

Depending on intended purpose, the present BET inhibitor or PARP inhibitor may be administered via any suitable routes, for example, oral or parenteral administration. Examples of parenteral administration include, but are not limited to, subcutaneous, mucosal, intratumoral, intravenous, intraarterial, or peritoneal injection. In one exemplary embodiment, the present BET inhibitor or PARP inhibitor is orally administered to the subject.

According to some embodiments of the present disclosure, compared to the treatment of BET inhibitor or PARP inhibitor, the combined treatment of BET inhibitor and PARP inhibitor exhibits a synergistic effect on tumors. According to some embodiments, compared with the subject unresponsive to the platinum-based therapy (e.g., cisplatin treatment), the combined treatment of BET inhibitor and PARP inhibitor significantly prolongs the survival (including overall survival and disease-free survival) of the subject responsive to the platinum-based therapy (e.g., cisplatin treatment).

According to various embodiments of the present disclosure, the pancreatic cancer may be a pancreatic adenocarcinoma or a pancreatic non-adenocarcinoma (e.g., a neuroendocrine pancreatic cancer). In one exemplary embodiment, the pancreatic cancer is PDAC.

The subject treatable with the present method is a mammal; for example, a human, mouse, rat, guinea pig, hamster, monkey, pig, dog, cat, horse, sheep, goat, cow, or rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Cell Culture

Four human PDAC cell lines were used in this study, including MIA-PaCa2, Panc-1, AsPC1, and BxPC3, in which the MIA-PaCa2, Panc-1 and AsPC1 comprises a KRAS mutation (i.e., KRAS mutant cell lines), and the BxPC3 has a wild-type KRAS gene (see, for example, Wen-Hsuan Chang et al., Cancer Lett. (2021), 517: 66-77; and Radu Pirlog et al., J Clin Invest. (2022), 132(14): e161454). The MIA-PaCa2 cells were cultured in RPMI1640 medium containing 10% fetal bovine serum (FBS) and 1% penicillin/streptomycin. The AsPC1, BxPC3 and Panc-1 cells were cultured in DMEM high glucose medium supplemented with 10% FBS and 1% penicillin/streptomycin.

### Cytotoxicity and Cell Proliferation Assays

Cytotoxicity and cell proliferation were determined by using 3-(4,5-dimethylthiazol-2-yl)-2,5-diphenyltetrazolium bromide (MTT) assay. Specifically, the MIA-PaCa2 and Panc-1 were seeded in a 96-well plate (5×10³ cells/well). 24 hours later, the BET inhibitor (mivebresib, JQ1 or AZD5153) and PARP inhibitor (rucaparib, veliparib or olaparib) were serially diluted and added to the cells. The cells were incubated at 37° C. for 48 hours. Then, 0.5 mg/ml MTT was added to the cells, followed by incubating at 37° C. for 3 hours. The resulting formazan crystals were dissolved using dimethyl sulfoxide (DMSO), and the absorbance was measured at 495 nm using a microplate reader. The cell proliferation ability was quantified as the optical density (OD) value. The cytotoxicity induced by the treatments was determined by calculating the percentage of viable cells (dividing the optical density of the drug-treated samples by that of the control samples).

### Drug Combination and Synergy Analyses

The combination index (CI) was determined by equation (I) to evaluate the interaction between drugs:

$$CI = \frac{{( D )}_{1}}{{( {D}_{x} )}_{1}} + \frac{{( D )}_{2}}{{( {D}_{x} )}_{2}} ; \qquad ( I )$$

in which (Dx)₁ represents the dose of the drug (D)₁ alone that inhibits the growth of cells by x %, and (Dx)₂ represents the dose of the drug (D)₂ alone that inhibits the growth of cell by x %. In the present study, the inhibitory effect (x %) was set to be 50%. A CI value less than 1 (CI<1) indicates a synergistic effect, a CI value of 1 (CI=1) indicates an additive effect, and a CI value greater than 1 (CI>1) indicates an antagonistic effect between drugs.

In operation, the PDAC cells were seeded in a 96-well plate (5×10³ cells/well), and incubated for 24 hours. The cells were then treated with various concentrations of BET inhibitor and PARP inhibitor. After 48 hours, MTT assay was used to determine cell number in accordance with the procedures described above. The synergy of the drug combination was assessed by determining the CI using software.

### Animal Model-Subcutaneous Tumor

The MIA-PaCa2 cells (2×10⁶) were suspended in 10 μL phosphate buffered saline (PBS), and then subcutaneously injected to NOD-SCID male mice (6-8 weeks old). After 14 days (the tumor size was about 100-150 mm³), PBS, mivebresib (1 mg/Kg), rucaparib (10 mg/Kg), or the combination of mivebresib and rucaparib (1 mg/Kg of mivebresib plus 10 mg/Kg of rucaparib) was orally administered to the mice five times per week, for three consecutive weeks. The tumor size was measured using caliper every 2-3 days, until the mice met humane endpoint criteria and were euthanized.

### Animal Model-Orthotropic Model

The shLacZ-MIA-PaCa2-Luc/GFP cells (2×10⁶; BIM-expressing cells) or shBIM-MIA-PaCa2-Luc/GFP cells (2×10⁶; BIM-knockout cells) were suspended in 5 μL PBS and 5 μL basement membrane matrix, followed by injected into the pancreas of NOD-SCID male mice (6-8 weeks old). After 14 days, PBS, mivebresib (1 mg/Kg), rucaparib (10 mg/Kg), or the combination of mivebresib and rucaparib (1 mg/Kg of mivebresib plus 10 mg/Kg of rucaparib) was orally administered to the mice five times per week, for three consecutive weeks.

The tumor growth was monitored using IVIS® imaging system, and the bioluminescent signal from cancer cells were analyzed by software to evaluate the treatment efficacy of monotherapy or combined therapy.

### Statistics

The results were analyzed by software, and presented as mean±standard error of the mean (mean±SEM). Two-tailed unpaired t-tests were performed to determine statistically significant differences between two groups. A p-value of ≤0.05 was considered significant. The levels of significance were denoted as follows: ns (not significant), *p≤0.05, **p≤0.01, ***p≤0.001, ****p≤0.0001 for the indicated comparisons.

### Example 1 Effect of BET Inhibitor and PARP Inhibitor on Pancreatic Cancer Cells

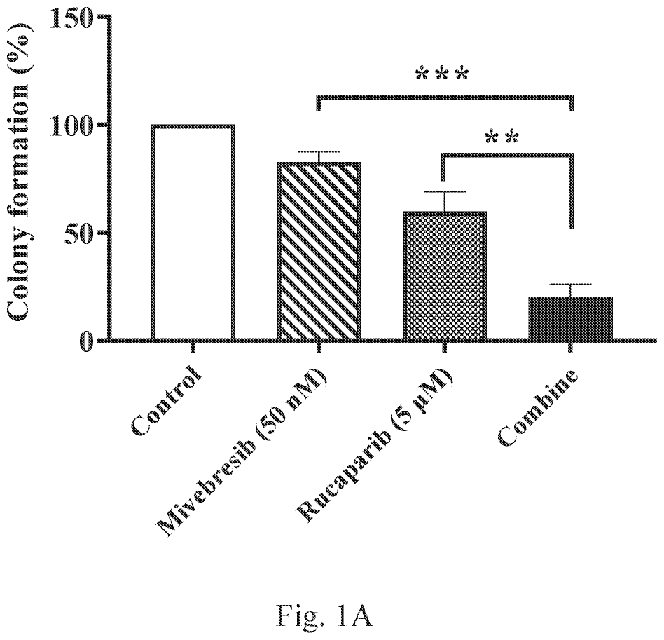

For the purpose of examining the effect of BET inhibitor and PARP inhibitor on pancreatic cancer cells, different BET inhibitors (including mivebresib, JQ1 and AZD5153) and PARP inhibitors (including rucaparib, veliparib and olaparib) were respectively added to MIA-PaCa2, Panc-1, AsPC1 and BxPC3 cells. 72 hours post-treatment, clonogenic assay and MTT assay were used to determine the colony formation and cell viability. The results were respectively depicted in FIGS. 1A, 1B, and Table 1.

According to the results of FIGS. 1A, 1B and Table 1, compared to the single treatment (i.e., mivebresib or rucaparib treatment), the combined treatment of mivebresib and rucaparib exhibited a synergetic effect on different pancreatic cancer cell lines (including MIA-PaCa2, Panc-1, AsPC1 and BxPC3 cells). The CI value calculated from MTT analytic results further confirmed the synergism between mivebresib and rucaparib (CI<1.0).

TABLE 1
IC₅₀ values of mivebresib and rucaparib
in specified pancreatic cancer cell line

| Cell line | Mivebresib | Rucaparib | Mivebresib + Rucaparib |
|---|---|---|---|
| MIA-PaCa2 | 259.77 | 2,444 | 13.38 |
| Panc-1 | 1,410 | 1,978 | 586.05 |
| AsPC1 | 191.27 | 2,363 | 96.61 |
| BxPC3 | 273.09 | 2,514 | 115.72 |

The synergistic effect were also observed in the combination of different BET and PRAP inhibitors (including the combination of JQ1 and rucaparib, the combination of mivebresib and veliparib, and the combination of AZD5153 and olaparib) (data not shown). It was noted that mivebresib and rucaparib induced the greatest synergistic effect among the tested BET and PARP inhibitors.

According to the results of fluorescent staining, flow cytometry and cell cycle arrest assays, the combined treatment of mivebresib and rucaparib induced mitochondrial instability and dysfunction that contribute to cell apoptosis and increase the percentage of sub-G1 phase (data not shown). The data of realtime-PCR and western blot further demonstrated that compared to the single treatment, the combined treatment of mivebresib and rucaparib synergistically increased the expression level of apoptosis gene BCL2L11 (BIM), and induced BCL2-related apoptosis pathway. These results indicated that mivebresib and rucaparib exerted an anti-tumor effect by inducing cell apoptosis through synergistically increasing BIM expression.

The data demonstrated that compared to the single treatment, the combined treatment of mivebresib and rucaparib inhibited the growth of pancreatic cancer cells in a synergistic manner.

### Example 2 Anti-Tumor Effect of Mivebresib and Rucaparib in Xenograft Mouse Model

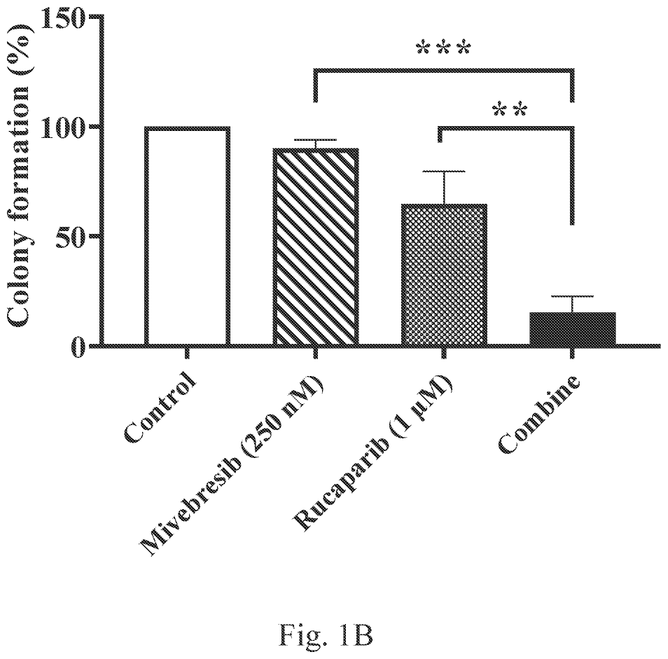

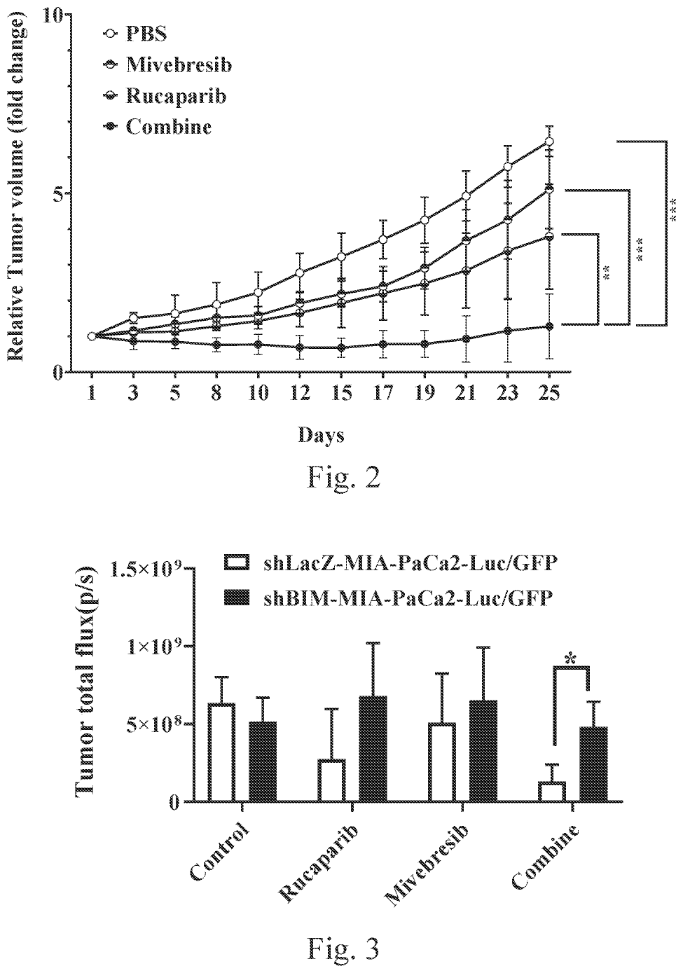

Two tumor models, including subcutaneous and orthotropic tumor models, were used in the example to evaluate the anti-tumor effect of mivebresib and rucaparib on pancreatic cancer. As described in “Materials and Methods”, the tumor-bearing mice were respectively administered with PBS, mivebresib, rucaparib, and the combination of mivebresib and rucaparib. The tumor sizes were monitored by caliper and IVIS® imaging system. The results were respectively depicted in FIG. 2 (subcutaneous tumor model) and FIG. 3 (orthotropic tumor model).

The data of FIG. 2 indicated that the treatment of mivebresib or rucaparib inhibited tumor growth in the subcutaneous tumor model. Compared to the single treatment, the combined treatment of mivebresib and rucaparib induced a more significant anti-tumor effect (FIG. 2). The mouse body weigh exhibited no significant changes during the course of the drug treatment (data not shown).

Similar anti-tumor effect was observed in the orthotropic tumor model, in which the bioluminescent signal from cancer cells was greatly reduced in the mice administered with combined treatment as compared to the mice administered with mivebresib or rucaparib treatment (FIG. 3). The data confirmed the synergistic effect of mivebresib and rucaparib on inhibiting tumor growth in the orthotropic pancreatic cancer model.

On the other hand, to investigate the role of the BIM gene in the synergistic effect of mivebresib and rucaparib, the shLacZ-MIA-PaCa2-Luc/GFP cells (BIM-expressing cells; serving as a control group) and shBIM-MIA-PaCa2-Luc/GFP cells (BIM-knockout cells) were respectively injected into the pancreas of mice. The tumor-bearing mice were respectively treated with specified drugs, and the tumor sizes were monitored by IVIS® imaging system. The data indicated that the bioluminescent signal from shBIM-MIA-PaCa2-Luc/GFP cells were obviously higher than that from shLacZ-MIA-PaCa2-Luc/GFP cells (FIG. 3), suggesting that the BIM gene plays an important role in the synergistic effect of mivebresib and rucaparib.

Additionally, the possibly systemic toxicity induced by mivebresib and rucaparib treatments was also evaluated in the example. According to the analytic results, the liver and kidney index determined by blood biochemical test, and the numbers of total neutrophil, red blood cell and platelet exhibited no significant variation amount the testing groups (data not shown), suggesting the safety use of the combined therapy by mivebresib and rucaparib.

The data of this example demonstrated that the combined treatment of mivebresib and rucaparib synergistically inhibited tumor growth in pancreatic tumor models, without inducing toxic or adverse results in the subjects.

### Example 3 Use of Mivebresib and Rucaparib in Patients Receiving Platinum-Based Therapy

Whether neoadjuvant chemotherapy (a chemotherapy that a cancer patient receives before the primary course of surgical treatment) would affect the overall survival of cancer patients was determined in the example. According to the results, the overall survivals of patient with or without cisplatin treatment were respectively 26.36 and 18.96 months (data not shown). Then, the patients receiving the platinum-based therapy were further divided into responder group and non-responder group. It was found that the overall survival and disease-free survival of the responder group were significantly longer than that of the non-responder group (data not shown).

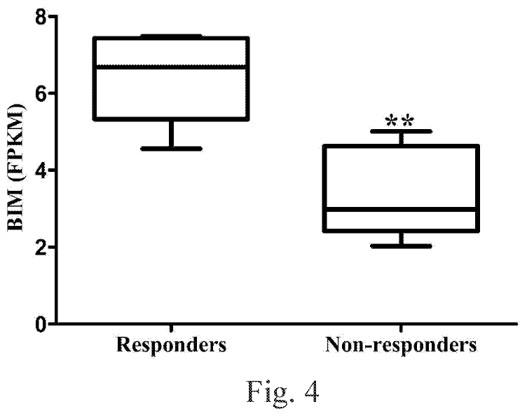

The samples derived from pancreatic cancer patients were subjected to sequence analysis. The results indicated that the expression of BIM gene in responders was significantly higher than that in non-responders (p=0.0015) (FIG. 4). The data suggested that the cancer patients who were responsive to platinum-based therapy and later developed resistance to the therapy may receive the combined treatment of BET inhibitor (e.g., mivebresib) and PARP inhibitor (e.g., rucaparib) as the secondary treatment, thereby addressing the problem of drug resistance.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating a pancreatic cancer in a subject who is responsive to a platinum-based therapy, comprising administering to the subject an effective amount of a BET inhibitor and an effective amount of a PARP inhibitor.

2. The method of claim 1, wherein the BET inhibitor is mivebresib, JQ1, or AZD5153.

3. The method of claim 2, wherein the BET inhibitor is the mivebresib.

4. The method of claim 1, wherein the PARP inhibitor is rucaparib, veliparib, or olaparib.

5. The method of claim 4, wherein the PAPR inhibitor is the rucaparib.

6. The method of claim 1, wherein the BET inhibitor and the PARP inhibitor are administered to the subject at a weight ratio of 1:10.

7. The method of claim 1, wherein the platinum-based therapy comprises the treatment of cisplatin, carboplatin, oxaliplatin, or nedaplatin.

8. The method of claim 7, wherein the platinum-based therapy comprises the treatment of the cisplatin.

9. The method of claim 1, wherein the subject is a human.
